(19) United States
(12) Patent Application Publication
(10) Pub. No.: US 2011/0160532 A1
(43) Pub. Date: Jun. 30, 2011

(54) ENDOSCOPIC INSTRUMENT

(75) Inventors: Rudolf HEIMBERGER, Oberderdingen (DE); Matthias LAMBERTZ, Bretten (DE)

(73) Assignee: RICHARD WOLF GMBH, Knittlingen (DE)

(21) Appl. No.: 12/977,192

(22) Filed: Dec. 23, 2010

(30) Foreign Application Priority Data
Dec. 29, 2009 (DE) 10 2009 060 718.8

(51) Int. Cl. A61B 1/018

(52) U.S. Cl. 600/106

(57) ABSTRACT

An endoscopic instrument includes an elongate guide element having at least one guide rail extending from a proximal to a distal end of the guide element. At least one working instrument, which is provided with an engagement element which fits with the at least one guide rail, engages with the at least one guide rail in a manner such that the working instrument is displaceable along the at least one guide rail.

### BACKGROUND OF THE INVENTION

The present invention relates to an endoscopic instrument.

The concept of new types of endoscopic methods, such as single-port techniques or NOTES (natural orifice translumenal endoscopic surgery) is to keep the access to the field of operation in a manner which is as non-traumatic and as small as possible. In order to be able to operate therapeutically, it is, however, necessary to apply several instruments simultaneously in a multifunctional manner. However, a more or less larger total diameter of the system results, depending on the number and design of these instruments to be used (e.g. gripper forceps, scissors, dissectors, HF-probes, ultrasound, laser, rinsing/suctioning, rotating knives, picture reproduction, etc.), by which means in turn a certain minimum size for the access is then necessary, which contradicts the aim of keeping the access as small as possible.

### BRIEF SUMMARY OF THE INVENTION

It is therefore an object of the present invention to improve an endoscopic instrument or a system of endoscopic instruments to the extent that the total system may be placed into the field of operational through a relatively small body opening.

The above object is achieved by an endoscopic instrument with the features specified in the independent claim(s). Preferred embodiments are to be deduced from the dependent claims, the subsequent description as well as the attached figures.

The basic concept of the present invention is based on the knowledge that many working instruments used in endoscopy, for the actual tools, require a larger cross-sectional area at the distal end than for the further distally extending shank. For example, the shank may be designed significantly more thinly than the distal end. A total system consisting of several such instruments may be led through a comparatively small access, if the instruments not all simultaneously, but one after the other, are led through the access, so that preferably always only one instrument with its region which is greatest in cross section, needs to simultaneously pass the access.

According to a preferred embodiment of the present invention, an endoscopic instrument or system of endoscopic instruments is now provided, which permits several correspondingly designed working instruments to be fixed relative to one another and to be advanced one after the other into the operation region in a defined manner, so that not all working instruments with their largest cross section need to pass the access simultaneously. For this, the endoscopic instrument according to a preferred embodiment of the present invention merely comprises an elongate guide element. This guide element serves for fastening and for guiding the working instruments, wherein in particular, the working instrument may be advanced into the operation region along the guide element. The elongate guide element thus represents the key element of the instrument or instrument system, according to the invention. The elongate guide element comprises at least one guide rail extending from the proximal to the distal end. Moreover, at least one working instrument is provided, which comprises an engagement element which is designed to fit with the guide rail, so that it may engage with the guide rail. The engagement element is then displaceable along the guide rail, so that the complete working instrument, after it has been introduced with the engagement element into the guide rail, may be displaced distally along the guide rail from the proximal end, in order to advance the working instrument into the operation region.

With an operation, according to the present invention, thus firstly the guide element is advanced through the access into the operation region and placed as is desired. Subsequently, one or more working instruments are advanced along one or more guide rails on the guide element, one after the other into the operation region. By way of the fact that the working instruments are engaged with the guide rail via the engagement elements, the individual working instruments are held on the guide element in a defined manner and furthermore are advanced in a defined manner, Accordingly, the working instruments may then also be pulled back proximally again along the guide rails one after another and removed from the operation region. Preferably, several guide rails are provided on the guide element, so that several working instruments may be fixed via the guide element, in defined positions to one another, and may be advanced on this guide element one after the other into the operation region.

At least one of the working instruments, seen in the longitudinal direction, comprises a middle section which has a smaller cross section than a distal section of the instrument. As already explained above, it is the case for example with forceps and cutting elements, that these at the distal end take up more space in cross section with respect to the instrument longitudinal axis, in order to be able to move gripper elements and/or cutting elements there. The shank extending proximally and departing from these actual working instruments, may be designed significantly more thinly, i.e. have a smaller cross section. Also, other working instruments such as picture transmission systems, endoscope optics, etc. at the distal end, often require a certain minimum size, in order to be able to functionally arrange the actual working instruments, objectives, openings or windows, such that optimal operation conditions are created. The proximally extending shanks may often however be designed significantly more thinly, since one does not require so much space in these, in order to be able to receive the systems required in the inside of the shank, for example actuation rods, fiber-optics, optical systems, rinsing channels, electrical leads etc. For this reason then, in the shank region of the working instrument, this is designed as thinly as possible, so that one may also lead several working instruments together through an as small as possible access. In order to able to lead the distal ends of the instruments which are widened in cross section, likewise through this access, the instruments are advanced one after the other along the guide element as described above, so that preferably always only one distal widening of the working instrument must simultaneously pass the narrowest location of the access.

As already discussed above, the guide element preferably comprises several guide rails, wherein these further preferably extend parallel to one another. Particularly preferably, the several guide rails are arranged distributed uniformly over the outer periphery of the guide element, so that several working instruments may be arranged distributed about the outer periphery of the guide element. An as large as possible distance between the individual instruments is provided by way of this arrangement, so that these may be advanced along the guide rails into the operation region and be used there for an operation in the envisaged manner, without these instruments colliding with one another or inhibiting one another. Preferably, all guide rails are designed equally. This has the advantage that the guide rails may be universally used and preferably the working instruments may be advanced by way of their engagement elements over any guide rails on the guide element. This permits the operator to place the individual working instruments on the guide element in a manner which he desires. The regular distribution of the guide rails over the outer periphery of the guide element supports this advantage, since with this arrangement, preferably all guide rails have the same distance or angular distance to one another and thus may be used in an infinite manner. However, it is also conceivable to design the guide rails in a different manner or to arrange them on the guide element differently distanced to one another, in order to permit a defined optimal placing of certain working instruments relative to one another.

The guide element may be designed in a rigid manner and thereby the guide element with the guide rails extends preferably in a straight manner. However, it is alternatively conceivable to design the guide element in a curved manner. Also a flexible and controllable design of the guide element with the guide rails is conceivable.

The guide rails are preferably designed as grooves in the peripheral surface or wall of the guide element. This has the advantage that the guide element on its outer periphery does not need to have any projecting elements or edges, which could lead to the damage of tissue on introducing the guide element.

Accordingly, the engagement element is preferably designed as an engagement projection in a manner such that it may engage into at least one of the engagement grooves. If preferably all engagement grooves are designed in an equal manner, then further preferably also all engagement projections are designed equally, so that the working instruments may be inserted into the engagement grooves in an infinite manner. Alternatively however, it is also conceivable to dimension certain engagement grooves and engagement projections or to design them with regard to shape, such that an engagement projection may only be applied into a certain engagement groove. Thus one may ensure that a certain working instrument is only inserted into a certain engagement groove and that certain working instruments are positioned to one another in a defined manner.

Further preferably, the at least one guide rail and the fitting engagement element are designed in a manner such the guide rail engages behind the engagement element in a direction transverse to the longitudinal axis of the guide rail or that the engagement element engages around the guide rail in a direction transverse to the longitudinal axis of the guide rail. In this manner, a securing or fastening in the direction transverse to the longitudinal direction of the guide rail is achieved, so that the engagement element and the working instrument which is connected to it, may be moved relative to the guide rail only in the direction of its longitudinal axis, but not transversely thereto. In particular, one prevents the engagement element being able to be pulled away from the guide rail in a direction transversely to the longitudinal axis of this, i.e. in particular normally to the longitudinal axis of the guide rail. In this manner, one prevents the guide rail and the working instrument from being able to separate during the operation. A separation is preferably possible exclusively in the manner such that the engagement element is pulled away from the guide rail in the longitudinal direction at the proximal end of this or that the engagement element is pulled in the proximal direction out of the guide rail.

If the guide rail is designed as an engagement groove, the engagement groove preferably at its outer side comprises an open gap, through which the engagement element engages into the engagement groove. Shown in cross section, this gap widens in the inside and correspondingly also the engagement element. For example, the gap and engagement element may be designed essentially T-shaped in cross section. In this manner, a guiding in the longitudinal direction of the guide rail is achieved and prevents the engagement element being able to be removed out of the guide rail transversely to the longitudinal direction. In preferred embodiments, it is conceivable to design the guide rail and engagement element such that the engagement element may be pivoted or rotated in or about the guide rail in a certain angular range about its longitudinal axis. Thus, a working instrument which is firmly connected to the engagement element or is designed as one piece with this, may be pivoted in a certain angular region about the longitudinal axis of the guide rail

The at least one working instrument is preferably a gripper instrument, a cutting instrument, a HF-instrument, a suction or rinsing channel, a picture transmission system and/or an illumination device. An endoscope may also be seen as a working instrument. A picture transmission system may be a video system or also an optical system for the direct picture transmission, as the case may be with the use of fiber-optics. Moreover, it is to be understood that the previously mentioned list of instruments is not conclusive, but rather all conceivable types of instruments which are required for an endoscopic operation, as working instruments, may be fastened on the guide element and advanced.

Moreover, the guide element in its inside may also comprise at least one channel extending from the proximal to the distal end. Such a channel may for example be applied as a suction channel or rinsing channel, but furthermore may also be applied or feeding or receiving further working instruments. Further preferably, an observation system and/or illumination system may be arranged in the inside of the guide element. Hereby, it may be the case of conventional endoscope optics, a video system or a system amid the use of fiber-optics for picture transmission and/or illumination. The arrangement of a optical system in the inside of the guide element has the advantage that with an introduction or an advance of the guide element, this optical system may be applied for observation, so that the guide element may be placed securely in the operation region under visual control.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

The invention is hereinafter described by way of example and by way of the attached figures. In the drawings:

FIG. 1 is a perspective view of a guide element of an endoscopic instrument according to a preferred embodiment of the present invention;

FIG. 2 is the guide element according to FIG. 1, with attached working instruments;

FIG. 3 is a lateral view of the instrument according to FIG. 2;

FIG. 4 is a sectioned view along line IV-IV in FIG. 3;

FIG. 5 is a sectioned view along line V-V in FIG. 3; and

FIG. 6 is a sectioned view along line VI-VI in FIG. 3.

### DETAILED DESCRIPTION OF THE INVENTION

Certain terminology is used in the following description for convenience only and is not limiting. Unless specifically set forth herein, the terms “a,” “an” and “the” are not limited to one element, but instead should be read as meaning “at least one.” The terminology includes the words used herein, derivatives thereof and words of similar import.

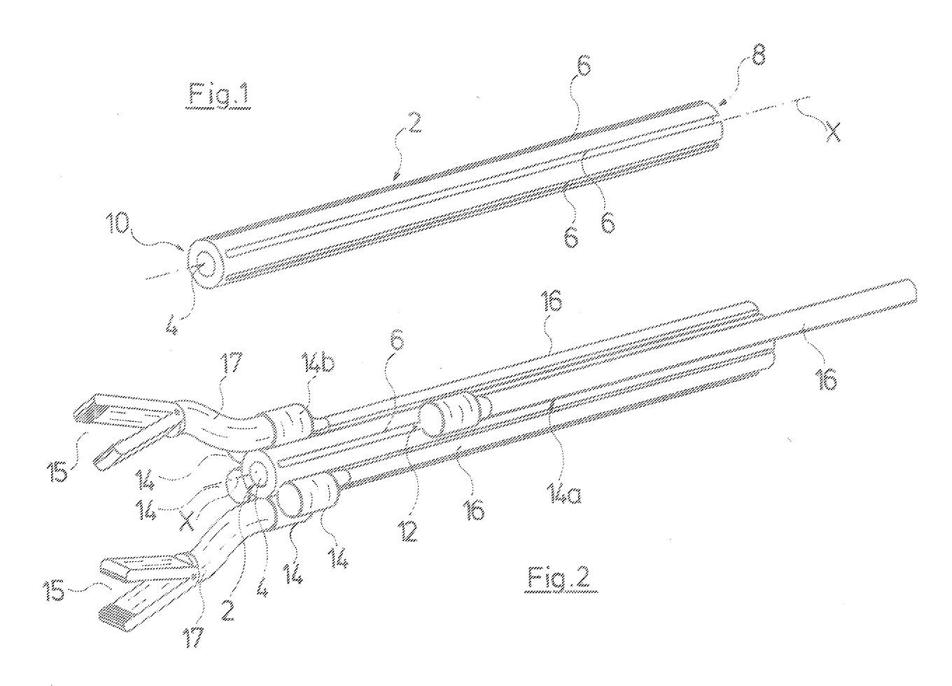

A key element of an endoscopic instrument according to a preferred embodiment of the present invention is a guide element 2, which is shown as an individual part in FIG. 1. The guide element 2 shown in FIG. 1 is designed in a tubular manner and in the middle has a central channel 4 extending along the longitudinal axis X. The channel 4 may serve for receiving working instruments or also an optical system. Such an optical system in the channel 4 may for example serve to advance the guide element 2 into the operation region under visual control. Alternatively or additionally, the channel 4 may also be applied as a rinsing channel or suctioning channel. In the example shown here, six guide rails 6 in the form of guide grooves and which extend parallel to the longitudinal axis X from the proximal end 8 to the distal end 10 of the guide element 2, are formed on the outer periphery of the guide element 2. Whereas the guide rails 6 are open towards the proximal end 8, they do not extend completely to the distal end 10 and are thus designed closed to the distal end 10.

The guide rails 6, as is to be recognized in FIG. 4-6, preferably have a circular cross section and are open to the outer periphery of the guide element 2 through a gap. An engagement element 12 in the form of an engagement projection engages into this gap. Thereby, this engagement projection has a cross section which corresponds to the cross section of the guide rail 6, so that a positive-fit engagement is created and in particular the engagement element 12 may only be inserted into the guide rail 6 from the proximal end 8, but is secured in this in the radial direction with respect to the longitudinal axis X, by way of the guide rail 6 engaging around the circular projection of the engagement element 12. Thus, a movement transverse to the longitudinal axis X is prevented.

The engagement elements 12 are fixedly connected to, or designed as one piece with, the working instruments 14. In this manner, the working instruments 14 (14, 14a, 14b) in each case may be inserted with the engagement element 12 into one of the guide rails 6 and may be advanced along this guide rail 6 towards the distal end 10 along the guide element 2. Five (5) working instruments 14 are already shown in the distal position in FIG. 2, whereas the working instrument 14a is still in an intermediate position, i.e., is represented during the advance to the distal end.

With regard to the working instruments 14, it may be the case of any instruments, which may be used for endoscopic operations, for example gripping instruments, cutting instruments, suction channels and rinsing channels, optical systems, endoscopes, HF-instruments, etc. Two working instruments as a forceps 15 are represented in the figures by way of example.

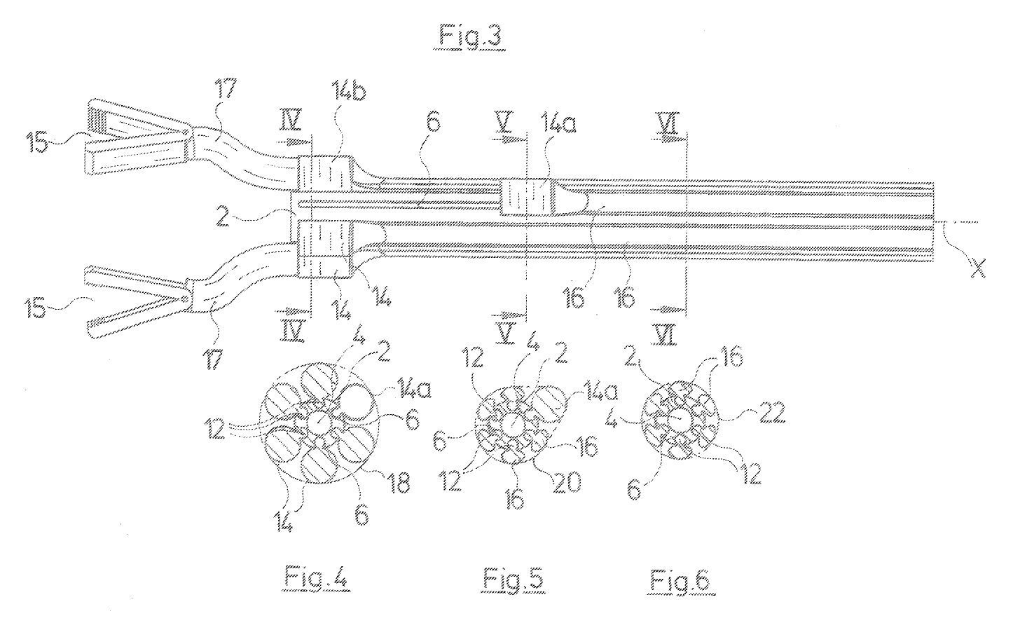

As may be recognized in FIGS. 2 and 3, the working instruments 14 at their distal end have a larger cross section with respect to the instrument longitudinal axis X than in the region of their remaining shank. As is to be seen for example by way of the instrument 14b, a larger cross section is required at the distal end, in order here to be able to ensure the desired functionality her, in the example, the movement ability of the forceps jaw 15. The shanks 16 which extend proximally towards the proximal end 8, in cross section have a smaller cross section. Only the necessary supply channels, actuation rods and likewise are arranged in these shanks In the example shown here, the working instruments 14 all have an outer shank tube which may have, for example, a forceps 15, scissors or likewise at its distal end. The actual working instrument in turn may be movably arranged in the inside of this shank tube, so that the working instrument may be moved proximally and distally. In this manner, the movement ability of each individual instrument during the operation is ensured independently of the guide rail 6. Simultaneously, it is also possible to design the instruments in a rotatable manner within the outer shank tubes, so that a change of the angular position of each individual working instrument 14 or of the actual instrument arranged in the shank tube of the working instrument 14 is possible.

With the shown forceps 15 used as an example, yet a shank section 17 is arranged between the distal end of the shanks 16 and the actual forceps jaw, and in the example shown here runs angled in a manner such that the forceps jaw is offset radially to the outside. The shank section 17 may be designed in a rigid manner, but it is also conceivable for the shank section 17 to be designed in a flexible or deflectable manner, in order to be able to move or position the forceps jaw of the forceps 15 in the operation field and in particular also to be able to position it in the radial direction with respect to the longitudinal axis X.

As is to be seen in FIG. 4, the cross section 18 in the region of the distal end of the complete arrangement is extremely large. The regions of the individual working instruments 14 which are widened in cross section are located there, so that here a maximal total cross section 18 results. As is shown in FIG. 6, a significantly smaller total cross section 22, which is the minimal cross section of the complete instrument or instrument system, results in the region of the shanks 16. According to a preferred embodiment of the present invention, it is then the case that the individual working instruments 14 are not advanced simultaneously through the access, but one after another on the guide element 2 in their respective guide rail 6, towards the distal end 10 and thus into the operation region. Thus, as is shown in FIG. 5, in the region of the access, through which the instrument is led into the body, a cross section 20 results, which is always only slightly enlarged compared to the minimal cross section 22. This cross section enlarges in each case always only by the amount of an individual working instrument 14 as is shown in FIGS. 3 and 5 by way of the working instrument 14a which has not yet been fully advanced. In this manner, it is possible to advance a number of working instruments 14 which at the distal end together define a large cross section 18, through a smaller cross sectional area 20 one after the other, into the operating region. Thus, despite a very small access, one may bring the complete instrument system consisting of numerous working instruments, through a single access into the operating region.

The working instruments 14 may be arranged in a sealed manner in the guide rails 6. Moreover, for the case that not all guide rails 6 are occupied with working instruments 14, a sealing of the unused guide rails 6 may also be possible by way of obturators. An exit of expanding body cavity gas from the body cavity is prevented by way of such sealing.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. An endoscopic instrument comprising an elongate guide element (2) including at least one guide rail (6) extending from a proximal (8) to a distal end (10) of the guide element, and at least one working instrument (14), provided with an engagement element (12) which fits with the at least one guide rail (6), engages with the at least one guide rail (6) in a manner such that the at least one working instrument (14) is displaceable along the at least one guide rail (6).

2. An endoscopic instrument according to claim 1, wherein the at least one working instrument (14), along a longitudinal direction (X), comprises a middle section (16) having a smaller cross section than a distal section thereof.

3. An endoscopic instrument according to claim 1, further comprising several guide rails (6) extending parallel to one another.

4. An endoscopic instrument according to claim 3, wherein one or more of the several guide rails (6) are formed as grooves in a peripheral surface of the guide element (2).

5. An endoscopic instrument according to claim 4, wherein the engagement element (12) is an engagement projection engaging into at least one of the several guide rails (6).

6. An endoscopic instrument according to claim 1, wherein the at least one guide rail (6) engages behind the engagement element (12) in a direction transversely to a longitudinal axis (X) of the at least one guide rail (6).

7. An endoscopic instrument according to claim 1, wherein the at least one working instrument (14) is one of a gripper instrument, a cutting instrument, a HF-instrument, a suction channel or rinsing channel, a picture transmission system and an illumination device.

8. An endoscopic instrument according to claim 1, wherein an inside of the guide element (2) comprises at least one channel (4) extending from the proximal to the distal end of the guide element.

9. An endoscopic instrument according to claim 1, wherein an observation system or an illumination system is arranged in an inside of the guide element (2).
